(19) United States
(12) Patent Application Publication
(10) Pub. No.: US 2005/0131501 A1
(43) Pub. Date: Jun. 16, 2005

(54) Apparatus and method for prevention and treatment of infection

(76) Inventor: Robert A. Rowland III, Houston, TX (US)

Correspondence Address: Jonathan P. Osha;ROSENTHAL & OSHA L.L.P., Suite 2800, 1221 McKinney, Houston, TX, 77010, US

(21) Appl. No.: 10/736,191

(22) Filed: Dec. 15, 2003

(51) Int. Cl. A61F 7/00; A61F 7/12

(52) U.S. Cl. 607/96

(57) ABSTRACT

A method for preventing and/or treating infection includes transitioning the temperature in a suspected area of infection based on an assessment of the existence and/or likelihood of infection in a localized area. Also, an apparatus for transitioning the temperature in a suspected area of infection, which includes a heat transfer element for transferring thermal energy to or from the suspected area of infection, and a thermal energy source for supplying thermal energy to the heat transfer element. The apparatus may also include one or more positioning elements and one or more temperature detecting elements. Activation of the apparatus may be manual or automated, and may rely on a feedback loop, or interaction with one or more external devices.

### BACKGROUND OF THE INVENTION

1. Field of the Invention

The invention relates generally to an apparatus and method for treating infection.

2. Background Art

Before the 16th and 17th centuries, there was no indication that disease could be caused by agents too small to be seen with the eye. The development of the microscope and the ability of ordinary people to describe their findings in minute detail created the opportunity for the discovery of infectious diseases and their subsequent management. It also encouraged the emergence of scholars in the arena of bacteriology as most of the original discoveries were made by relatively uneducated individuals with an inquisitive nature but little else.

Necessity has always encouraged innovation and so commercial activities were also in the forefront of this newly developing area with the subsequent transfer of ideas to human disease. Throughout the latter part of the 19th century, the ideas now critical to the area of infection control gradually developed. The principles were well established by the early 20th century and although the current strategies seem complex, they are simply advances on these original protocols.

Pasteur was the first to use the word ‘disease’ to describe a condition caused by an infective agent. His work involved the spoilage of beer and wine which he ascribed to the growth of undesirable microorganisms. Microorganisms were proven to also cause disease in animals with the discovery of the bacterium responsible for anthrax in cattle.

With the development of the Germ Theory of Disease (microbes cause disease) the bacterial agents responsible for a large number of human diseases were described and preventive strategies involving hygiene and, where possible, immunization were developed. It is now known that our bodies have three levels of defense against disease: the epithelial surfaces, cellular response, and the adaptive immune system. These three defense levels use physical, chemical and biochemical agents to act against disease pathogens. They act in order of level, for example if a pathogen breaches the epithelial or body surface level, the cellular level responds.

Despite the extensive array of pathogens encountered in the environment, infection is a relatively rare event. Frontline defense at epithelial surfaces, such as skin and oral mucosa, involves physical, chemical, and biochemical agents and is often able to intercept most pathogens and prevent their entry into the body.

After the first line of defense, the body has additional cellular defenses within its circulation. Since many pathogens possess mechanisms for evading the primary and secondary natural defenses, a third line of defense, the adaptive immune system, is important for controlling infection and eliminating the pathogen.

The first two natural defense levels can be breached by repeated assaults from a given pathogen because immunological memory is confined to the adaptive system. This means the body has an immunological memory which helps it deal successfully with repeated exposure to infectious agents through the development of targeted antibodies and immune cells. Cellular defenses within the adaptive immune system are directed by lymphocytes.

The immune system deals with pathogen infections in different ways. The strategy it uses depends on the nature of the organism and the pathology of the disease. Antibodies are made by the body's immune system and neutralize, deactivate or destroy harmful agents such as microorganisms and their toxins. Various response mechanisms are triggered by different forms of infection, and the response to a viral or fungal infection may differ in some respects from the response to a bacterial infection.

For pathogens that do not enter host cells (e.g. bacteria), antibodies neutralize the toxin and block adhesion of the toxin to target cells of the body. For pathogens that invade host cells (e.g., viruses), the antibodies typically opsonise the invaded cells (prepare them for ingestion by other immune cells) and a complement-mediated lysis (rupture) and removal by phagocytosis (engulfing by specialized immune cells) will typically occur. This form of immune response is often accompanied by pain, redness, swelling and heat, all of which are attributable to increased blood supply to the infected area.

During infection, blood supply to an infected area is increased for a number of reasons. A primary reason is that this facilitates the translocation of immune cells (e.g. white blood cells) to the area of infection. This increased blood supply often manifests itself as an inflammation in the area of infection. Three major changes that occur during inflammation are: (i) an increase in the diameter of blood vessels (dilation) and in the rate of blood flowing through them; (ii) an increased impermeability (resistance to penetration) of the local capillaries; and (iii) escape of serum and white blood cells into the inflamed area.

Although inflammation will typically signify an increase in vessel diameter, early phases of inflammation may include a brief period of vasoconstriction (restriction in vessel diameter). Any period of vasoconstriction may, to some extent, restrict blood flow to healthy tissues, as well as to any pathogens in the area of restricted blood flow. This decreased blood flow may result in suffocation of healthy tissue in the area of infection, possibly leading to cell death. Furthermore, lymphatic channels may be blocked in early stage inflammation, thereby localizing the inflammatory reaction to the affected region. Cell wall junctions will typically become permeable to certain white blood cells which are able to then enter the dilated vessels and quickly move to the affected area to fight the infection.

Although the normal immune response is sufficient to overcome and clear most infections, for certain pathogens the normal immune response is ineffective. Furthermore, those with compromised immune systems may have trouble fighting infection internally and must relay on external assistance in order to overcome an infection. Drugs are the most common approach when the body needs assistance in overcoming an infection. Numerous specific and non-specific drugs have been developed over the years, having varying levels of effectiveness

In recent years, certain pathogens, including bacteria, have been observed to develop a resistance to various medications commonly used fight them. For instance, overuse and overprescription of antibiotics has created many antibiotic-resistant strains of these pathogens. Although new drugs are being developed to address such resistant strains, it is feared that these pathogens will continue to develop resistances to new drugs as they begin to find widespread use. Furthermore, many viruses are known to mutate or otherwise evolve in ways which make them more resistant to the body's immune system, more effective infectious agents, and/or more resistant to antiviral drugs. Accordingly, there exists a need for a method and apparatus for fighting infection that does not rely on drugs.

### SUMMARY OF THE INVENTION

one embodiment, a method for preventing and/or inhibiting infection is disclosed. The method according to such an embodiment includes rapidly transitioning the temperature in a suspected area of infection and assessing the suspected area for infection, then discontinuing the transitioning of the temperature and reassessing the suspect area for infection. In one embodiment, the transitioning of temperature may be repeatedly performed and discontinued until a favorable assessment is achieved.

In one embodiment, the invention is an apparatus for the prevention and/or treatment of infection. Such an apparatus includes a heat transfer element and a thermal energy source. In one embodiment, the invention will also include a temperature detector. Furthermore, embodiments of the invention may also include a positioning member and/or temperature detector. In one embodiment, the invention will also include one or more inputs and/or outputs for communicating or otherwise interacting with associated devices.

Other aspects and advantages of the invention will be apparent from the following description and the appended claims.

### BRIEF DESCRIPTION OF DRAWINGS

FIG. 1 shows one embodiment of an apparatus for treating infection.

FIG. 2 shows one embodiment of an apparatus for treating infection.

### DETAILED DESCRIPTION

Numerous pathogens have been observed to exhibit variations in physiology and/or life cycle patterns due to variations in temperature. These variations may affect a pathogen's ability to initiate or perpetuate an infection. Furthermore, the temperature of the host's tissue surrounding a pathogen may not only affect the physiology and/or life cycle pattern of the pathogen, but may also affect the tissue's and the host's susceptibility to the pathogen.

Although not a limitation on the invention, one or more physiological mechanisms may play a role in the efficacy of the invention in the treatment and/or prevention of infection. Possible physiological effects that the invention may have on a pathogen include, but are not limited to, decreasing the pathogen's rate of replication, denaturing one or more proteins of the pathogen, and/or increasing a pathogen's susceptibility to one or more mechanisms of the host's immune system. The invention may also affect the mobility of the pathogen within the host, as well as the supply of nutrients to the pathogen. These effects may occur as a result of the constriction of blood vessels, and concordant decrease in blood flow into and out of the target area. Furthermore, embodiments of the invention may lessen the damage to healthy tissues caused by vasoconstriction and decreased blood flow to the affected area. For instance, a rapid, transient decrease in temperature may lower the metabolic needs of healthy cells in the affected area, thereby lessening the likelihood that such cells would “suffocate” and possibly die as a result of localized vasoconstriction.

In one embodiment, the invention relates to a method for inhibiting infection by rapidly decreasing the local temperature of a suspected area of infection until a predetermined temperature is reached. Once the predetermined temperature is reached, cooling is discontinued and the suspect area is assessed to determine the effectiveness of the cooling.

In one embodiment, the invention relates to a method for inhibiting infection by rapidly increasing the local temperature of a suspected area of infection until a predetermined temperature is reached. Once the predetermined temperature is reached, heating is discontinued and the suspect area is assessed to determine the effectiveness of the heating.

In one embodiment, the invention relates to a method for inhibiting infection by rapidly changing the local temperature of a suspected area of infection within a predetermined temperature range. Once the transitioning is complete, the target area is permitted to warm or cool to the normal temperature of the tissue and the suspect area is assessed to determine whether further temperature transitions are necessary. Changing of the temperature of the target area may include rapid heating followed by rapid cooling, or rapid cooling followed by rapid heating.

In one embodiment, the invention relates to a method for inhibiting infection by changing the local temperature of a suspected area of infection for a predetermined length of time. This predetermined length may vary based on the type of tissue targeted, the suspected cause of the infection, and/or any other criteria known in the art.

The method may be practiced on any living organism (hereinafter “subject”). The degree and rapidity of temperature transition may be varied based upon a number of factors. These include, but are not limited to, the type of subject, the condition of the subject, the perceived severity of infection or perceived likelihood of infection, the apparatus used for implementation, and/or the sensitivity of the tissue to changes in temperature.

Although the transitioning of temperature is described herein as being regulated on the basis of treatment criteria including one or more of temperature, rapidity, and duration, other treatment criteria may also be a factor in the transitioning. For example, the subject's perceived level of discomfort and/or the results of a biopsy of the suspected area may comprise treatment criteria upon which the temperature transitions may be based. In one embodiment, the subject's level of discomfort, as reported by the subject or otherwise assessed, may be a factor in determining the discontinuation of the rapid transitioning of the temperature of a suspect area. Furthermore, any other treatment criteria known in the art may be used, individually or in conjunction with other treatment criteria, as a basis for regulating the temperature transitions.

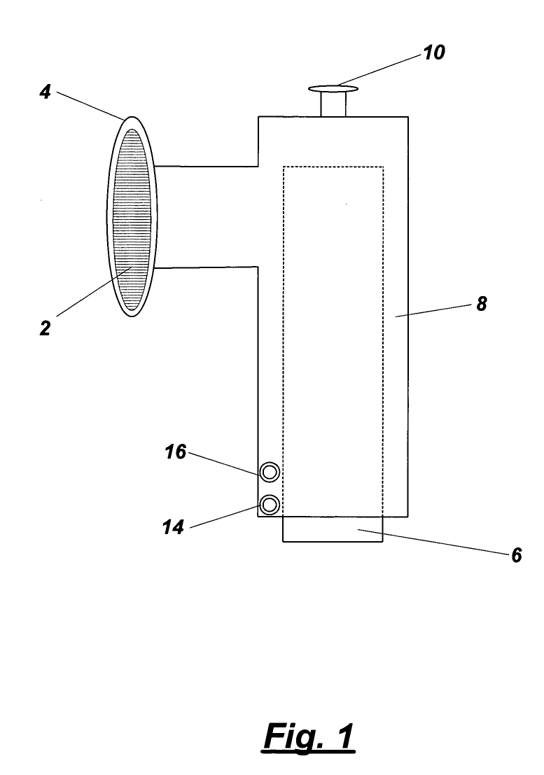

In one embodiment, the invention relates to an apparatus for inhibiting infection. As shown in FIG. 1, such an apparatus will typically include a heat transfer element 2, an insulating element 4, a thermal energy source 6, and a positioning element 8. The heat transfer element 2 may vary in configuration, and may be of any configuration known in the art to be effective for transitioning the temperature of a localized area of tissue. The thermal energy source 6 will be operatively connected to the heat transfer element 2. In one embodiment, the thermal energy source 6 will be replaceable and/or renewable, so that the thermal energy source 6 may be recharged (e.g., one or more thermal energy components is renewed) and/or replaced if and when depleted.

Although not required in some embodiments, the insulating element 4 will be configured to inhibit heat exchange between the heat transfer element 2 and the surrounding environment. For instance, if the invention is configured for use on a limb, the heat transfer element 2 may be curved to facilitate contact with the curved surface of the limb, and accordingly the insulating element 4 may be similarly curved, to maintain close contact with the heat transfer element 2, and thereby more effectively insulate the heat transfer element 2 from the surrounding environment.

In one embodiment, the thermal energy source 6 may be integrated with the heat transfer element 2. For example, the thermal energy source 6 may form a unitary structure with the heat transfer element 2. In such an embodiment, the invention will include an activation element 10 for activating the thermal energy source, in order to facilitate a desired temperature transition in the heat transfer element 2. In such an embodiment, the activation element 10 may be configured to be in an activated state for a predetermined period with each activation. In an alternative embodiment, the activation element 10 may be configured to activate for only as long as an operator maintains activation (e.g. activated for as long as a button is depressed, etc.). The activation element 10 may be of any type known in the art including, but not limited to, a button or switch. In another embodiment, the invention may include one or more mechanisms for detecting the temperature of the heat transfer element 2 and/or target tissue and may self-activate and/or deactivate based on this detection.

In one embodiment, the thermal energy source 6 may be separate from the heat transfer element. For instance, the thermal energy source 6 may comprise one or more chemicals which, separately or in combination with one another, may be applied to the heat transfer element 2 in order to achieve a desired temperature of the heat transfer element 2.

In one embodiment, the invention may communicate with one or more other devices to regulate the initiation, deactivation, and/or degree of heat transfer to the target area. For instance, one or more devices may monitor the temperature of the target area and/or any other vital statistic of the subject and communicate and/or interact with the invention on the basis thereof. One or more inputs 14 and/or outputs 16 may be provided for such interaction. These may be located anywhere on the device, and may be of any type known in the art.

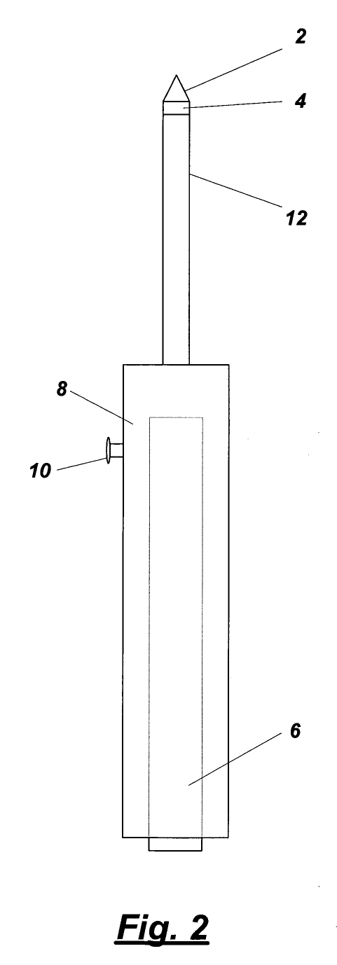

The embodiment of FIG. 2 is a probe-like configuration which may advantageously facilitate positioning of the heat transfer element 2 in closer proximity to a suspected area of infection. For example, the heat transfer element 2 may penetrate into the suspected area in order to more directly and/or effectively create a temperature transition in the target area. In such a configuration, the heat transfer element 2 may be located at any point along the arm 12 of the embodiment.

In use, the heat transfer element 2 will be positioned a predetermined distance from a suspected location of infection. For external application, where the embodiment of FIG. 1 may be more appropriate, the heat transfer element may be positioned in close proximity to, or in contact with, the skin of a subject. Based on the degree of temperature transition desired, the proximity to the suspected location of infection may be manipulated. For instance, by applying varying degrees of pressure to the skin using the embodiment of FIG. 1, the proximity to the suspected area and/or the efficiency of heat transfer may be altered, thereby affecting the degree of temperature variation occurring in the suspected area. Once application of the heat transfer element 2 is terminated, the target area and/or subject may be assessed to determine whether a second application is required. Such assessment may include an evaluation of the perceived and/or reported level of discomfort. Based on such an evaluation, treatment may be terminated or repeated. In one embodiment, the detection of a sudden, sharp increase in discomfort followed by a gradual decrease in discomfort, will indicate that the procedure has been successfully executed and that no further treatment of the suspect area is required.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A method for inhibiting infection, comprising:
(a) causing a rapid temperature change in a suspected area of infection;
(b) discontinuing the causing of the rapid temperature change; and
(c) assessing the suspected area for occurrence of infection.

2. The method of claim 1, wherein the causing of step (a) is continued until a predetermined temperature is reached.

3. The method of claim 2, wherein the predetermined temperature is sustained for a predetermined period of time, prior to step (b).

4. The method of claim 1, wherein the causing of step (a) occurs until any discomfort in the suspected area decreases to a predetermined level.

5. The method of claim 1, wherein the assessing comprises evaluating a subject's level of discomfort.

6. The method of claim 5, wherein treatment is terminated if the evaluating indicates a rapid increase in discomfort followed by a gradual decrease in discomfort.

7. The method of claim 1, further comprising repeating steps (a)-(c) if the assessing in step (c) indicates that infection may still occur.

8. An apparatus for inhibiting infection, comprising:
a heat transfer element having a surface configured to be positioned in close proximity to a suspected area of infection; and
a thermal energy source for altering a temperature of the surface of the heat transfer element until a predetermined temperature is reached.

9. The apparatus of claim 8, wherein the thermal energy source forms an integral unit with the heat transfer element.

10. The apparatus of claim 8, wherein the surface of the heat transfer element is configured to a shape of a target area.

11. The apparatus of claim 8, further comprising a temperature detector.

12. The apparatus of claim 11, wherein the temperature detector regulates activation of the thermal energy source.

13. The apparatus of claim 8, further comprising at least one selected from an input and an output, for communicating with at least one other device.

14. The apparatus of claim 8, further comprising an insulating element.

15. The apparatus of claim 8, further comprising a positioning element.

16. The apparatus of claim 8, wherein the thermal energy source is separately replaceable.

17. The apparatus of claim 8, wherein the thermal energy source includes an input for renewal of at least one component of the thermal energy source.

18. A method for using an apparatus for inhibiting infection, comprising:
positioning a surface of a heat transfer element in close proximity to a suspected area of infection; and
activating the apparatus.

19. The method of claim 18, further comprising discontinuing activation of the apparatus once a treatment criteria is met.

20. The method of claim 18, wherein the activating is initiated by a temperature detector.

21. The method of claim 18, wherein the activating occurs for a predetermined period.

22. The method of claim 18, wherein the activating is initiated by one or more external devices in communication with the apparatus.

23. The method of claim 18, further comprising discontinuing activation of the apparatus based on reaching a predetermined temperature in a target area.

24. The method of claim 18, further comprising discontinuing activation of the apparatus based once a predetermined temperature of a target area is maintained for a predetermined amount of time.
